(19) United States
(12) Patent Application Publication
(10) Pub. No.: US 2016/0100796 A1
(43) Pub. Date: Apr. 14, 2016

(54) PLURAL TASK FITTING

(71) Applicant: Cochlear Limited, Macquarie University (AU)

(72) Inventor: Sean LINEAWEAVER, Gig Harbor, WA (US)

(21) Appl. No.: 14/879,617

(22) Filed: Oct. 9, 2015

Related U.S. Application Data
(60) Provisional application No. 62/062,218, filed on Oct. 10, 2014.

(51) Int. Cl. A61B 5/00; A61B 5/12; A61N 1/36

(52) U.S. Cl. CPC A61B 5/4851; A61N 1/36032; A61B 5/121; A61B 5/7475

(57) ABSTRACT

A method, including subjecting the recipient to a plurality of groups of tasks, respective groups of tasks corresponding to respective sets of parameters by which the device will be fitted, obtaining data indicative of respective listening effort associated with the respective groups of tasks based on performance of the recipient of the respective groups of tasks, at least partially fitting the hearing prosthesis to the recipient based on the obtained data.

### CROSS-REFERENCE TO RELATED APPLICATIONS

This application claims priority to Provisional U.S. Patent Application No. 62/062,218, entitled PLURAL TASK FITTING, filed on Oct. 10, 2014, naming Sean LINEAWEAVER of Gig Harbor, Wash., as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from hearing loss typically receive an acoustic hearing aid. Conventional hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve. Cases of conductive hearing loss typically are treated by means of bone conduction hearing aids. In contrast to conventional hearing aids, these devices use a mechanical actuator that is coupled to the skull bone to apply the amplified sound.

In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound.

Many devices, such as medical devices that interface with a recipient, have structural and/or functional features where there is utilitarian value in adjusting such features for an individual recipient. The process by which a device that interfaces with or otherwise is used by the recipient is tailored or customized or otherwise adjusted for the specific needs or specific wants or specific characteristics of the recipient is commonly referred to as fitting. One type of medical device where there is utilitarian value in fitting such to an individual recipient is the above-noted cochlear implant. That said, other types of medical devices, such as other types of hearing prostheses, exist where there is utilitarian value in fitting such to the recipient.

### SUMMARY

In accordance with an exemplary embodiment, there is a method, comprising subjecting the recipient to a first task, subjecting the recipient to a second task of a different type than the first task, wherein the first task and the second task draw from the same cognitive domain of the recipient and at least partially fitting a device to the recipient based on results of the first and second task.

In accordance with another exemplary embodiment, there is a non-transitory computer-readable media having recorded thereon, a computer program for executing at least a portion of a method of fitting a hearing prosthesis to a recipient, the computer program including code for obtaining data indicative of respective listening effort by the recipient associated with a respective groups of tasks subjected to the recipient based on performance of the recipient of the respective groups of tasks, the respective groups of tasks corresponding to respective sets of parameters by which the device will be fitted, and code for at least partially fitting the hearing prosthesis to the recipient based on the obtained data.

In accordance with yet another exemplary embodiment, there is a system for at least partially fitting a device to a recipient, comprising, a processor and a device configured to visually display a plurality of words to the recipient, wherein the system is configured to receive input from the recipient indicative of a choice of one or more of the plurality of words, the processor is configured to receive information indicative of the input from the recipient and determine whether the choice corresponds to one or more words in a sentence previously presented to the recipient, wherein the one or more words corresponds to fewer words than that in the sentence previously presented to the recipient and the processor is configured to select a fitting parameter based on the determination and based on information pertaining to word perception of the sentence previously presented to the recipient.

In accordance with yet another exemplary embodiment, there is a method, comprising executing a subjective process to obtain a plurality of potential fitting parameters, and after executing the subjective process, executing an objective process to select a subset of the plurality of potential fitting parameters obtained in the subjective process, and at least partially fitting a device to a recipient using at least one of the fitting parameters of the selected subset.

In accordance with yet another exemplary embodiment, there is a method of fitting a hearing prosthesis to a recipient, the method comprising subjecting the recipient to a group of task, obtaining data indicative of respective listening effort by the recipient associated with the respective groups of tasks subjected to the recipient based on performance of the recipient of the respective groups of tasks, the respective groups of tasks corresponding to respective sets of parameters by which the device will be fitted, and at least partially fitting the hearing prosthesis to the recipient based on the obtained data.

In accordance with yet another exemplary embodiment, there is a system for at least partially fitting a device to a recipient, comprising a processor, and a device configured to visually display a plurality of words to the recipient, wherein the system is configured to receive input from the recipient indicative of a choice of one or more of the plurality of words, the processor is configured to receive information indicative of the input from the recipient and determine whether the choice corresponds to one or more words in a sentence previously presented to the recipient, and the processor is configured to select a fitting parameter based on the determination and based on information pertaining to word perception of the sentence previously presented to the recipient.

### BRIEF DESCRIPTION OF THE DRAWINGS

Embodiments are described below with reference to the attached drawings, in which:

FIG. 1 is a perspective view of an exemplary hearing prosthesis in which at least some of the teachings detailed herein are applicable;

FIG. 2 presents an exemplary flowchart for an exemplary method according to an exemplary embodiment;

FIG. 3 presents another exemplary flowchart for an exemplary method according to an exemplary embodiment;

FIG. 3 presents another exemplary flowchart for an exemplary method according to an exemplary embodiment;

FIG. 4 presents another exemplary flowchart for an exemplary method according to an exemplary embodiment;

FIG. 5 presents another exemplary flowchart for an exemplary method according to an exemplary embodiment;

FIG. 6 presents another exemplary flowchart for an exemplary method according to an exemplary embodiment;

FIG. 7 presents another exemplary flowchart for an exemplary method according to an exemplary embodiment; and

FIG. 8 presents an exemplary functional schematic of a system according to an exemplary embodiment.

### DETAILED DESCRIPTION

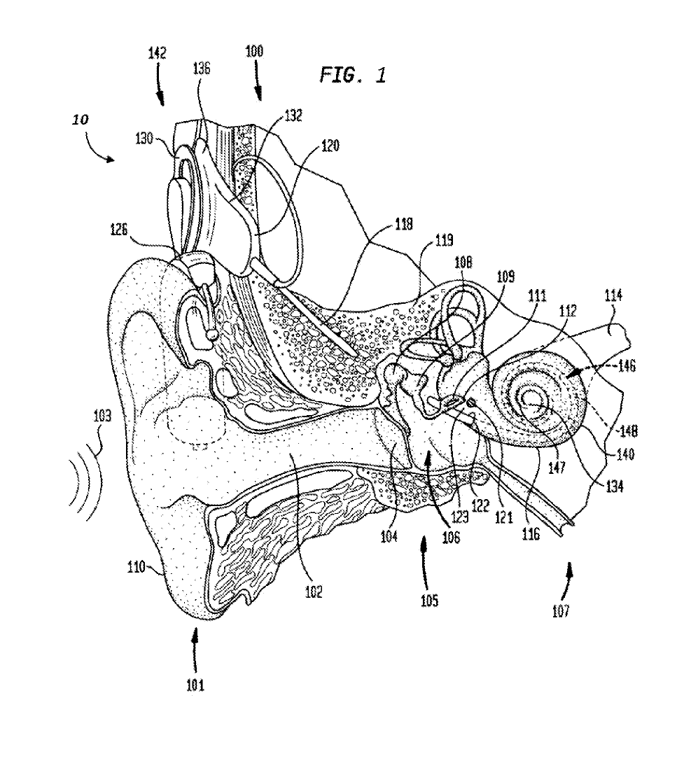

FIG. 1 is a perspective view of a cochlear implant, referred to as cochlear implant 100, implanted in a recipient, to which some embodiments detailed herein and/or variations thereof are applicable. The cochlear implant 100 is part of a system 10 that can include external components in some embodiments, as will be detailed below. It is noted that the teachings detailed herein are applicable, in at least some embodiments, to partially implantable and/or totally implantable cochlear implants (i.e., with regard to the latter, such as those having an implanted microphone). It is further noted that the teachings detailed herein are also applicable to other stimulating devices that utilize an electrical current beyond cochlear implants (e.g., auditory brain stimulators, pacemakers, etc.). Additionally, it is noted that the teachings detailed herein are also applicable to fitting and/or using other types of hearing prosthesis, such as by way of example only and not by way of limitation, bone conduction devices, direct acoustic cochlear stimulators, middle ear implants etc. Indeed, it is noted that the teachings detailed herein are also applicable to so-called hybrid devices. In an exemplary embodiment, these hybrid devices apply both electrical stimulation and acoustic stimulation to the recipient. Any type of hearing prosthesis to which the teachings detailed herein and are variations thereof can have utility can be used in some embodiments of the teachings detailed herein

The recipient has an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

In an exemplary embodiment, subsequent implantation of the cochlear implant 100, the recipient can have the cochlear implant 100 fitted or customized to conform to the specific recipient desires/to have a configuration (e.g., by way of programming) that is more utilitarian than might otherwise be the case. This procedure is detailed below in terms of a cochlear implant by way of example. It is noted that the below procedure is applicable, albeit perhaps in more general terms, to other types of hearing prosthesis, such as by way of example only and not by way of limitation, bone conduction devices, direct acoustic cochlear implants, sometimes referred to as middle-ear-implants, etc. Also, the below procedure can be applicable, again albeit perhaps in more general terms, to other types of devices that are fitted to a recipient.

The cochlear implant 100 is, in an exemplary embodiment, an implant that enables a wide variety of fitting options that can be customized for an individual recipient. Embodiments of the teachings detailed herein and/or variations thereof can be applied to a heterogeneous population of cochlear implant recipients, where a given recipient has utilitarian value that is maximized with a different set of parameters of the cochlear implant, which can maximize speech reception and/or recipient satisfaction.

In an exemplary embodiment, the fitting methods detailed herein are executed in conjunction with a clinical professional, such as by way of example only and not by way of limitation, an audiologist, who selects a set of parameters, referred to herein as a parameter map or, more simply, a MAP, that will provide utilitarian sound reception for an individual recipient. That said, in an alternate embodiment, the fitting methods detailed herein are executed without a clinical professional, at least with respect to some of the method actions detailed herein. Additional details associated with the cooperation and lack of cooperation of a clinical professional are detailed below.

An exemplary embodiment entails fitting a device, such as a cochlear implant, to a recipient based at least in part on listening effort of the recipient. More specifically, an exemplary embodiment entails obtaining data associated with listening effort for a given set of parameters of the device, obtaining data associated with listening effort for another set of parameters of the device, comparing the two sets of data, and selecting the set of parameters that indicates that the recipient had an easier time of listening based on the data. In an exemplary embodiment, the selected set of parameters is selected for the set of parameters that result in the least effortful listening experience for the recipient. That said, in an alternate embodiment, this is but one of the factors that play into the selection of the set of parameters.

More specifically, an exemplary embodiment entails obtaining information related to listening effort by an interactive dual task (which can include additional tasks, as long as it includes at least two tasks). The dual task includes a task associated with speech understanding (speech perception, speech recognition), below referred to as a “listening task,” and a task associated with memory accuracy, below referred to as a “memory task.” In an exemplary embodiment, these tasks draw from the same cognitive domain of the recipient. By “draw from the same cognitive domain of the recipient,” it is meant that the dual tasks require the use of the same perceptual domains. This is as contrasted to tasks requiring the use of two separate perceptual domains, such as by way of example only and not by way of limitation, a dual task comprising a visual task and a listening task, a dual task comprising a language task (or comprehension task) and a listening task, or a proprioceptive task and a listening task. Thus, in an exemplary embodiment, listening effort is gauged or otherwise determined based on working memory (i.e., the cognitive process which includes the executive and attention control of short-term memory, and provide for the interim integration, processing, disposal and retrieval of information). That is, in an exemplary embodiment, the evaluations of respective characteristics of working memory associated with respective sets of parameters are made, and, based on the characteristics of the working memory, a set of parameters is selected based on the working memory valuation.

In an exemplary embodiment, the tasks of the dual tasks are tasks that, statistically (based on a general population of which the recipient is apart) or individually (based on an analysis of the specific recipient) will not be performed “simultaneously” or within close temporal proximity with efficiency at least generally corresponding to that which would result from the performance of those tasks individually, or at least substantially temporally separated. More specifically, in an exemplary embodiment where a dual task encompasses “task A” and “task B,” task B is a task that is not performed as efficiently as it would otherwise be if it was a task that drew from a different cognitive domain and/or if performed separately from task A. This is as contrasted to a dual task encompassing “task X” and “task Y”, where task Y is a task that is performed at least substantially as efficiently as it would be if it was a task that drew from a different cognitive domain as task X. By way of example only and not by way of limitation, task A and task B of the dual tasks are tasks that interfere with one another because the tasks compete for the same class of information processing resources in the recipient's brain. In this regard, task B is a task that is more effortful when practiced with task A because of the cognitive interference. In an exemplary embodiment, task A is a listening task, and task B is a memory task. In an exemplary embodiment, task A can be a task that can be performed at about the same efficiency at various levels of listening effort. That is, increased listening effort relatively minimally impacts the performance of that task. Conversely, task B is a task that cannot be performed at about the same efficiency at various levels of listening effort (at least for a given recipient and/or for a statistically pertinent sample of a pertinent population). That is, task B is a task the nature of which performance thereon will decrease as listening effort increases.

By way of example only and not by way of limitation, task B is a task that is at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 175%, about 200%, about 225%, about 250%, about 275%, about 300%, about 350%, about 400%, about 450%, about 500% or more or any value or range of values therebetween in 1% increments, more effortful when performed in conjunction with task A than it would otherwise be if not performed in conjunction with task A.

In view of the above, an exemplary embodiment entails fitting a device, such as a hearing prosthesis (e.g., cochlear implant), to a recipient thereof, based on an assessment of listening effort (also referred to as ease of listening and/or auditory cognitive load). The assessment of listening effort is based on results of performance of the recipient in executing the dual tasks (those tasks drawing from the same cognitive domain, as noted above). It is noted that in at least some embodiments, listening effort can be gauged by determining ease of listening, auditory processing/task load, cognitive load, etc. Accordingly, in at least some embodiments, listening effort includes and/or is analogous to the aforementioned phrases.

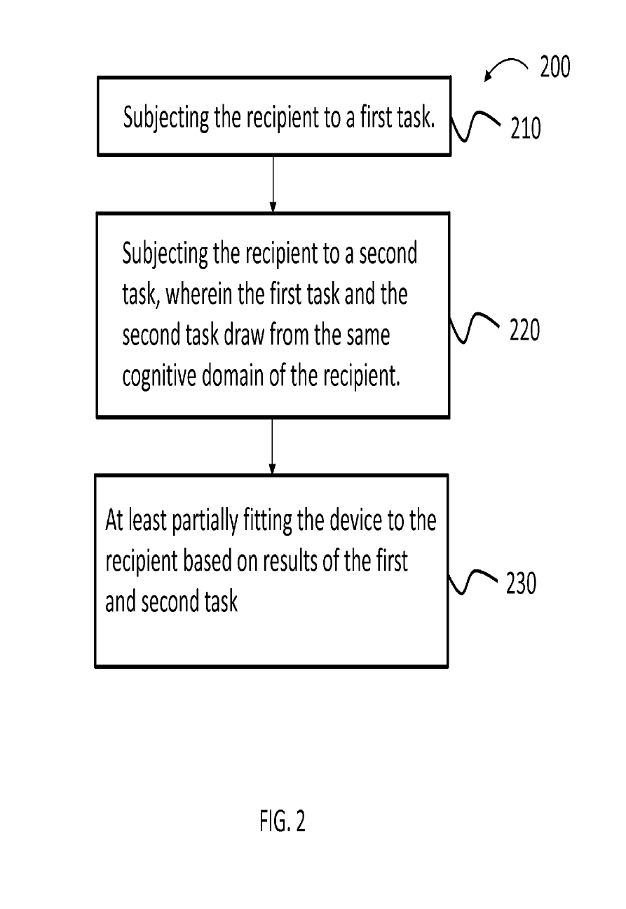

FIG. 2 presents an exemplary flowchart for an exemplary method 200 according to an exemplary embodiment. As can be seen, method 200 includes method actions 210, 220 and 230. Method action 210 entails subjecting the recipient to a first task. In an exemplary embodiment, the first task is a sentence recognition test. More specifically, the recipient of the cochlear implant (or other hearing prosthesis) is presented with a plurality of sentences that are received by the cochlear implant in such a manner that the cochlear implant evokes a hearing percept based on sentences. In an exemplary embodiment, the speaker or the like is utilized to generate the plurality of sentences. The generated plurality of sentences is subsequently captured by a sound capture device (e.g., microphone) of the cochlear implant 100. In an alternative embodiment, the plurality of sentences is provided to the cochlear implant 100 by a wired connection and/or a wireless connection, bypassing the sound capture device. Any device, system and/or method that can enable the cochlear implant 100 to evoke a hearing percept such that method 200 can be executed can be utilized in at least some embodiments.

In an exemplary embodiment, the recipient is exposed to ten (10) sentences, although fewer or more sentences can be used in alternate embodiments. In an exemplary embodiment, 2, 3, 4, 5, 6, 7, 8, 9, 10, 11, 12, 13, 14, 15, 16, 17, 18, 19, 20 or more sentences or any value or range of values therebetween in increments of 1 are subjected to the recipient. Any number of sentences that can enable the teachings detailed herein and/or variations thereof to be practiced can be utilized in at least some embodiments.

Is further noted that the phrase “subjecting the recipient” includes both a scenario where a clinician or an automated device presents tasks or otherwise instructs the recipient to execute tasks, and a scenario where the recipient initiates the tasks himself or herself and/or where the recipient initiates a session where the tasks are presented (which can be the case in the example of an automated interactive system, some of the details which are discussed below).

In an exemplary embodiment of method action 210, after one or more respective sentences are exposed to the recipient, the recipient indicates what he or she perceived as being exposed to him or her. This can be done in a strictly serial manner—exposure of sentence 1, indication of perception of sentence 1, exposure of sentence 2, indication of perception of sentence 2, exposure of sentence 3, indication of perception of sentence 3, and so on. That said, in an alternate embodiment, the sequence can be in a different manner (e.g., two sentences can be exposed to the recipient, and the recipient can then indicate the perceptions of the two sentences, etc.). It is further noted that with respect to the term “sentence,” it does not mean that a complete and/or complex or even proper sentence must be utilized. It can be a sentence fragment. Indeed, the sentences can be 2, 3, 4, 5, 6, 7, 8, 9 or 10 or more word sentences. Accordingly, as used herein, the term “sentence” means a string of words having utilitarian value with respect to the teachings detailed herein. Any method of ascertaining the extent to which a recipient understands speech or otherwise perceives speech can be utilized in at least some embodiments, providing that the teachings detailed herein and are variations thereof to be practiced.

As noted above, method action 210 entails the recipient indicating what he or she perceived as being the sentence. In an exemplary embodiment, the indication corresponds to oral repetition of the given sentence. In an alternative embodiment, the indication corresponds to the recipient writing down the sentence. Alternatively and/or in addition to this, in an alternative embodiment, the indication corresponds to the recipient selecting a sentence from a group of sentences presented to the recipient in a visual manner. Additional details of such indication are described below. It is noted that any device, system and/or method that will enable an indication of perception of a sentence can be utilized in at least some embodiments.

Referring back to FIG. 2, it can be seen that method 200 further includes method action 220, which entails subjecting the recipient to a second task of a different type than the first task. In an exemplary embodiment, the second task is a memory task, and thus of a different type than that of the first task (which, as noted above, in this exemplary embodiment, is a speech perception/recognition task). Consistent with the teachings detailed above, despite the fact that the second task is a different type of task than the first task, the second task is drawn from the same cognitive domain of the recipient.

In an exemplary embodiment, the second task can entail the recipient remembering and/or mentally retaining the last word of each of the sentences presented in the first task. More specifically, the recipient of the cochlear implant, who has been presented with the plurality of sentences that are received by the cochlear implant in such a manner that the cochlear implant evokes a hearing percept based on the sentences, as noted above in method 210, remembers the last word that he or she perceived in each sentence as a result of the hearing precept evoked by the cochlear implant. After the plurality of sentences are presented to the recipient, or at least some of them, and after the recipient has presented an indication of perception of those sentences, or at least some of them, the recipient then indicates what he or she remembers with respect to those sentences. For example, after the recipient is exposed to ten (10) sentences (or fewer or more as noted above), and after the recipient provides the indications of the hearing perceptions for those sentences, the recipient then indicates what he or she remembers about those sentences. By way of example only and not by way of limitation, the recipient memorizes specific words from the various sentences, and then indicates the words that he or she remembers from the sentences. In an exemplary embodiment described above, the recipient is tasked to remember the last word in the sentence, and the recipient indicates what he or she remembers as the last word of each sentence. That said, in an alternate embodiment, the recipient can be tasked to instead remember the first word in the sentence and/or a word in between the first word and the last word and/or a plurality of words within the sentence. Any method of a memory test that can enable the teachings detailed herein and/or variations thereof to be practiced can be utilized in at least some embodiments.

As inferred above, the order in which the recipient indicates what he or she remembers relative to the actions of indicating what he or she perceived as being exposed to him or her can be different in some embodiments. In an exemplary embodiment, this can be done in a strictly grouped serial manner—exposure of sentence 1, indication of perception of sentence 1, exposure of sentence 2, indication of perception of sentence 2, exposure of sentence 3, indication of perception of sentence 3, and so on (e.g., for all of the sentences of the group) followed by indication of the remembered word from sentence 1, indication of the remembered word from sentence 2, indication of the remembered word from sentence 3, and so on (e.g., for all of the sentences of the group). That said, in an alternate embodiment, the sequence can be in a different manner (e.g., after two or more speech perception tasks are executed for respective tasks, two or more respective memory tasks are executed, followed by two or more speech perception tasks followed by two or more respective memory tasks etc.). Accordingly, method 200 can be practiced such that method action 210 and method action 220 are executed in an interleaved fashion. Any order of implementing the tasks of method action 210 and 220 that can enable the teachings detailed herein and/or variations thereof to be practiced can be utilized in at least some embodiments.

As noted above, method action 220 entails the recipient indicating what he or she remembers about each sentence. In an exemplary embodiment, the indication corresponds to oral repetition of the word and/or words at issue in the sentence. In an alternative embodiment, the indication corresponds to the recipient writing down the word or words. Alternatively and/or in addition to this, the indication corresponds to the recipient selecting a word or words from a group of words presented to the recipient in a visual manner. Additional details of such indication are described below. It is noted that any device, system and/or method that will enable an indication of what is remembered about a given sentence can be utilized in at least some embodiments.

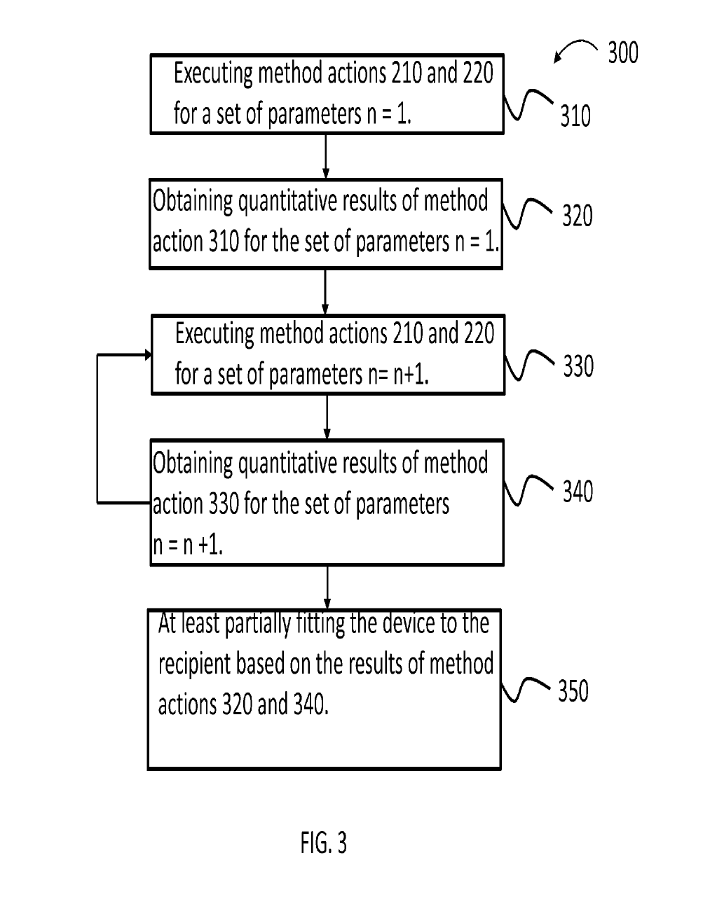

Method 200 further includes method action 230, which entails at least partially fitting the device to the recipient based on results of the first and second task. In an exemplary embodiment, in the case of a cochlear implant or other hearing prosthesis, this can entail selecting a set of parameters that correspond to parameters where the recipient had relative success, relative to other sets of parameters, in the tasks of method actions 210 and 220, and adjusting or otherwise configuring the cochlear implant to operate utilizing that set of parameters. In this vein, an exemplary embodiment includes method 300, where FIG. 3 presents an exemplary flowchart for such method. As can be seen, method 300 includes method action 310, which entails executing method actions 210 and 220 for a set of parameters of the cochlear implant which are labeled n=1 (a first set of parameters). In an exemplary embodiment, this can entail providing a speech perception task and a memory task for ten sentences (or more or fewer) as detailed above.

Method 300 further includes method action 320, which entails obtaining quantitative results of method action 310 for the set of parameters n=1 (the first set of parameters). In an exemplary embodiment, this entails scoring the recipient's performance on the tasks presented in method action 310. By way of example only and not by way of limitation, the recipient can be scored based on the number of sentences correctly understood by the recipient with respect to the speech understanding tasks/speech perception tasks, and scored based on the number of words correctly recalled/correctly remembered with respect to the memory tasks. For example, if the recipient correctly perceived 7 of 10 sentences, and remembered 8 aspects of the 10 sentences (e.g., 8 of the last words of the 10 sentences), the recipient would have a score of 70% on the perception tasks and a score of 80% on the memory tasks. That said, in an alternative embodiment, method 300 is executed by simply obtaining results of method action 310 (i.e., an exemplary flowchart for this alternate method would correspond to that depicted in FIG. 3, but not include the words “quantitative”). That is, in at least some embodiments, it is not required to obtain quantitative results. Other types of results can be obtained. By way of example only and not by way limitation, in at least some embodiments, the results can entail obtaining the recipient feedback to the tasks (e.g., information indicative of what the recipient perceives as being heard, information indicative of what the recipient remembers etc.). That said, in at least some embodiments, quantitative results can be obtained outside the method. Is further noted that in an alternate embodiment, method action 320 can be executed by simply obtaining the quantitative results. That is, it is not necessary to actually score the recipient to execute method action 320. Instead, in this exemplary embodiment, it is only necessary to obtain the scores (i.e. the scoring can be performed outside of the method).

It is noted that in an exemplary embodiment, at least some of the methods detailed herein utilize quantitative testing/scoring as opposed to and/or in addition to qualitative testing (or judgment)/scoring.

It is noted that method actions 310 and 320 can be executed by a clinical professional, such as an audiologist or the like. That said, as will be detailed below, in an alternative embodiment, some or all of these methods can be executed in an automated or automatic manner. Still, according to at least some embodiments, method actions 310 and 320 will be executed in a clinical setting, wherein the tester (e.g., audiologist) presents sentences to the recipient that are used by the hearing prosthesis to evoke a hearing percept, and instructs or otherwise has the recipient attempt to repeat or otherwise identify (e.g., by writing or the like—again described in greater detail below) what he or she perceives as being heard. This is part of method action 310. Method action 310 further includes the tester instructing the recipient or otherwise having a recipient identify what he or she remembers as a given word in the perception test (e.g., the final word of each sentence). The tester then scores the recipient as to how many words and/or sentences were accurately perceived, and how many words and/or word groups were accurately remembered. This is method action 320. Accordingly, the relationship between the tester and the recipient is interactive, in that the tester presents the speech and memory material to the recipient, and the recipient identifies the answers, followed by the tester scoring the recipient's feedback.

It is noted that there exists the possibility that a recipient can “fail” or otherwise receive a wrong score on the memory task if the recipient incorrectly perceived a keyword on the listening task. More specifically, if the recipient did not accurately perceive the “memory” word during the listening task, the recipient naturally will not be able to identify that word as part of the memory recall exercise. Accordingly, the only way to “pass” the memory task is to “pass” the speech perception task/listening task. For example, if the listening task entails the recipient to perceive the sentence “everybody loves the red dog,” and the recipient instead perceives the sentence as being “everybody loves red bob,” when asked to remember the last word of the sentence, a recipient will correctly remember the word “bob,” and thus the recipient will state the word “bob” instead of the word “dog.” That is, even though the recipient correctly remembers what he or she perceived as being the word or words at issue, the recipient will be scored as giving an incorrect answer on the memory test, at least without some of the teachings detailed herein.

Accordingly, an exemplary embodiment includes a memory task that is based upon the words perceived in the listening task. This is as opposed to basing the memory task on the actual words presented to the recipient in the listening task. That is, by way of example only and not by way of limitation, in the scenario where the recipient perceived the word “bob” instead of the word “dog,” the memory task would be scored based upon whether the recipient could recall the word “bob” instead of the word “dog.” Accordingly, an exemplary embodiment includes obtaining the results of a listening task, and also obtaining the results of the memory task based on the obtained results of the listening task (as opposed to obtaining the results of the memory task based on the subject matter subjected to the recipient in the listening task). Thus, an exemplary embodiment presents a memory task where it is not necessary to accurately perceive words presented during a listening task. That is, an exemplary manner in which method action 310 is executed is by presenting a plurality of sentences to the recipient during a listening task, and subsequent to presentation of respective sentences, having the recipient identify what the recipient perceived as the respective sentence, and subsequent to presentation of the plurality of sentences, and subsequent to the identification of what the recipient perceived as the respective sentence, having the recipient identify what the recipient perceived as the respective memory word and/or words (e.g., the last word of the sentence, the first word of the sentence, etc.) of the respective sentences of the plurality of sentences.

One exemplary method of decoupling an erroneous perception of speech from the memory task is to have the recipient indicate what he or she perceived as being heard as the sentence presented to the recipient during the listening task. One exemplary embodiment entails having a recipient vocalize or otherwise “repeat back” a sentence during the listening task and memorializing in some manner what the recipient vocalized, which may only entail identifying the words that were different from that presented to the recipient (where the baseline is that the recipient will correctly perceived words not indicated as being different). The results of the memory task can be compared to the aforementioned memorialization to avoid or otherwise discount any issues associated with misperception of words during the listening task. In an alternative embodiment, the recipient writes down a sentence during the listening task, or at least writes the word that is identified as the memory word (or words).

An alternative embodiment, a “multiple choice” regime can be utilized to decouple an erroneous perception of speech from the memory task. By way of example only and not by way of limitation, interactive media can be utilized to accomplish this task. For example, the listening task can be performed by presenting the recipient a list of textual sentences on a video screen from which he or she can choose. The recipient can then choose from the list of sentences that particular sentence that he or she perceived. This will enable the recipient to provide a definitive answer to what he or she perceived without any attenuation (or with relatively less attenuation), or at least with less attenuation compared to the clinician ascertaining what the recipient perceived. (This is especially the case if the recipient has a speech impairment or otherwise has speech production issues that cause the recipient to speak less clearly than normal members of a given population, or even if the recipient speaks clearly, speech is laborious for the recipient, thus negating or otherwise reducing any fatigue associated with having to speak, which could influence the results of the listening tasks and/or the memory tasks—this is also utilitarian with respect to a semi-automated or formally automated system that utilizes speech recognition software or the like as detailed below). From this input from the recipient, the memory word(s) will correspond to the perceived words, as opposed to the words of the sentence presented to the recipient (which may be the same in the case of perfect perception), and thus any incorrect perceptions will not be carried over into the memory task.

In an exemplary embodiment, the memory recall component can be detached or otherwise separated from reliance upon accurate perception in the listening task by audibly presenting, for example 10 sentences, such that the hearing prosthesis evokes a hearing percept based on those 10 sentences, and having the recipient select sentences from a list of sentences. For example, each time a sentence is provided to the recipient, a screen can display 2, 3, 4, 5, 6, 7, 8, 9, and/or 10 or more sentences. The recipient can touch the sentence (or more accurately, touch the screen where the sentence is located) to select a given sentence. Subsequently, the recipient can be presented with a screen that includes by way of example only and not by way of limitation, 10, 20 or 30 or 40 or 50 or more words, presented in alphabetical order or the like, from which the recipient chooses the memory words. Accordingly, in an exemplary embodiment, a close-set approach can be utilized to obtain information indicative of what the recipient remembers. It is noted that the above can be implemented utilizing an automated device, such as a computer or the like. Additional details of such are presented below. Also from the above, it can be seen that exemplary embodiments can be implemented where it is not necessary for the recipient to provide a verbal repetition or verbal indication of what he or she perceives as being heard, thus providing utilitarian value to recipients with speech production problems or otherwise who become fatigued through speaking.

It is noted that the above exemplary scenario details utilizes a video screen or the like or some other interactive technology, where a touchscreen of like is utilized utilized, where the recipient touches the text of the sentence that he or she perceives as corresponding to that which was presented to him or her during the listening task. That said, in an alternate embodiment, a paper list or the like can be presented to the recipient, where the recipient selects the sentence from the list (e.g., circles the sentence stabs the list with a pencil (potentially utilitarian for children to keep their interest or otherwise make the tasks less “test like”)). It is further noted that the above examples can be applied to the memory task as well. That is, the recipient can select from a list of text words presented on the screen (or paper). Any device, system and/or method that can enable the recipient to convey data to the clinician (or other entity) indicative of what the recipient perceives when being subjected to the listening tasks and/or what the recipient remembers when being subjected to the memory tasks can be utilized in at least some embodiments.

Additional details of some exemplary embodiments of how such a regime can be implemented are described in additional detail below. However, at this time, it is noted that in view of the above, exemplary method actions can entail obtaining the results of the listening task which includes obtaining an incorrect response of a listening sub-task by the recipient and obtaining the results of the memory task based on the obtained results of the listening task by comparing a respective memory sub-task result to the obtained incorrect response. That is, the memory task can be executed with success even though an incorrect answer was provided on the listening task. Also in view of the above, an exemplary method action can entail obtaining the results of the listening task by identifying a respective perceived word that is different from a respective actual word presented to the recipient in the listening task, and obtaining the results of the memory task based on the obtained results of the listening task by comparing a respective remembered word to the respective perceived word.

Continuing with reference to FIG. 3, method 300 further includes method action 330, which entails executing method actions 210 and 220 for a set of parameters n=n+1 (e.g., a second set of parameters) of the cochlear implant different than those used to execute method action 310. Method 300 further includes method action 340, which entails obtaining quantitative results of method action 330. In an exemplary embodiment, this entails scoring the recipient's performance on the tasks presented in method action 330 as noted above with respect to method action 320. That said, in an alternate embodiment, method action 340 can simply entail obtaining results of method action 330, in the manners akin to those noted above.

After executing method action 340, method 300 returns to method action 330, where method action 330 is executed for a new set of parameters n=n+1 (e.g., a third, fourth, fifth, sixth, seventh, eighth, ninth, tenth, etc., set of parameters). The loop is repeated for as many respective sets of parameters as deemed utilitarian (which can be more than the ten just noted by way of example).

After executing method actions 330 and 340 for the sets of parameters deemed utilitarian for which to execute those methods, method 300 proceeds to method action 350, which entails at least partially fitting the bone conduction device based on the results of method actions 320 and 340. By way of example only and not by way of limitation, in an exemplary embodiment, the obtaining quantitative results obtained from the method actions 320 and 340 can be compared to one another, and the set of parameters can be selected from amongst the group of sets of parameters “n” based on the comparison. For example, if a set of parameters yields the highest scores with respect to the memory task and the speech perception task, that set of parameters can be utilized to fit the cochlear implant. That said, in an alternative embodiment, a set of parameters can be selected that does not correspond to parameters that yield the highest scores with respect to the memory task and the speech perception task, at least if there is a reason to do so. Other criteria can be utilized, such as, by way of example only and not by way limitation, a weighting regime. With brief reference to the above-noted method of utilizing perceived words from the listening task in the memory task, it is noted that in an exemplary embodiment, method action 350 entails fitting the device (e.g., a hearing prosthesis) to the recipient based on congruence between the perceived respective memory word and/or words of the respective sentence of the plurality of sentences and the remembered respective word and/or words of the respective sentence of the plurality of sentences. Thus, an exemplary embodiment entails fitting a hearing prosthesis utilizing the above-noted method of accounting for misperception of words to avoid false negatives in the memory tasks.

Additional details of the selection of the sets parameters based on the results of method actions 310 and 330 are described below. It is noted at this time that any device, system or method of selecting the set of parameters deemed utilitarian to fit the cochlear implant therewith based on results of method actions 310 and 330 can be utilized in at least some embodiments.

It is further noted that the actions of method 300 do not have to be practiced in a serial manner. For example, method 300 can be practiced by executing method action 330 before executing method action 320. Still by further example, method action 340 can be practiced after executing method action 330 for all executions or for some executions. Any order of execution of the method actions, including an interleaving of sub-actions of the method actions, that can enable the teachings detailed herein that are variations thereof to be practiced, can be utilized in at least some embodiments.

In view of the above, an exemplary method entails determining a listening effort, or at least obtaining data indicative of a listening effort, based on the obtained results obtained in method actions 320 and 340 and/or based on any other method that will result in a listening effort being ascertained or otherwise gauged. Along these lines, in an exemplary embodiment, the tasks of method actions 210 and 220 are such that and/or are presented in such a manner that relative performance on the memory task will decrease with relative increased listening effort for a given set of parameters. That is, the harder it is to listen/the more effort required to be expended with listening, the harder it will be for the recipient to remember the words of the sentences presented to him or her. Accordingly, an exemplary embodiment entails utilizing the quantitative results of the memory task to ascertain a level of listening effort that is expended with the cochlear implant when (or if it was) configured with a given set of parameters. In an exemplary embodiment, the cochlear implant is fitted with a set of parameters that correspond to those that indicate that the recipient had an easier time listening/the listening was less effortful relative to that of one or more or all of the other sets of parameters.

In view of the above, by implementing an effort task or otherwise basing the fitting of a device based on effort (e.g., listening effort in the case where the device is a hearing prosthesis) associated with a given task to which the device has utilitarian value (e.g., perceiving speech), the listening effort test can be utilized to compare different settings of the device, and provide information that can aid a clinician in determining a utilitarian setting for that device. More specifically, with respect to the hearing prosthesis in general and the cochlear implant in particular, the listening effort test can be used to compare different settings, such as different cochlear implant programs/maps, or even different situations, such as different processing strategies, different programming parameter values, different processors and/or different listening conditions. In an exemplary embodiment, the setting and/or situation having most utilitarian value can be the one that is both least effortful for listening and has the highest results with respect to speech perception and/or some weighted combination of the two.

In an exemplary embodiment, method actions 310 and 330 of method 300 are objective tasks. Method action 210 entails identifying first fitting parameters that are, for the recipient, indicative of least effortful listening relative to other fitting parameters (e.g., from amongst the group of the set of parameters “n” with reference to method 300 above). Method action 220 entails identifying second fitting parameters that are, for the recipient, indicative of most perceivable speech relative to other fitting parameters (again, by way of example, from amongst the group of the set of parameters “n” with reference to method 300 above). Ultimately, a set of parameters, corresponding to a single subset of the sets of parameters “n,” is selected based on the identification of the first fitting parameters and the second fitting parameters (and, in some instances, based on other information), and the medical device (e.g., hearing prosthesis) is fitted based on the selected parameters.

The listening tasks and memory tasks detailed herein can enable a method that entails identifying fitting parameters that are, for the recipient, indicative of least effortful listening relative to other fitting parameters and indicative of most perceivable speech relative to other fitting parameters. The hearing prosthesis is then fitted based on the identified fitting parameters. Further, the listening tasks and memory tasks detailed herein can enable a method that entails identifying respective degrees of effortful listening for respective fitting parameters and identifying respective degrees of perceivable speech for the respective fitting parameters. A correlation is identified between various sets of parameters and the degrees of effortful listening and the degrees of perceivable speech, and the hearing prosthesis is then fitted based on the degrees of effortful listening and the degrees of perceivable speech.

In an alternate embodiment, the hearing prosthesis is fitted according to a method based on information relating to listening effort, but where the prosthesis is fitted with a set of parameters that do not correspond to that which would result in the least effortful listening because another phenomenon may override the selection of that set of parameters. Accordingly, it is enough in at least some methods to take into account listening effort when selecting the set of parameters.

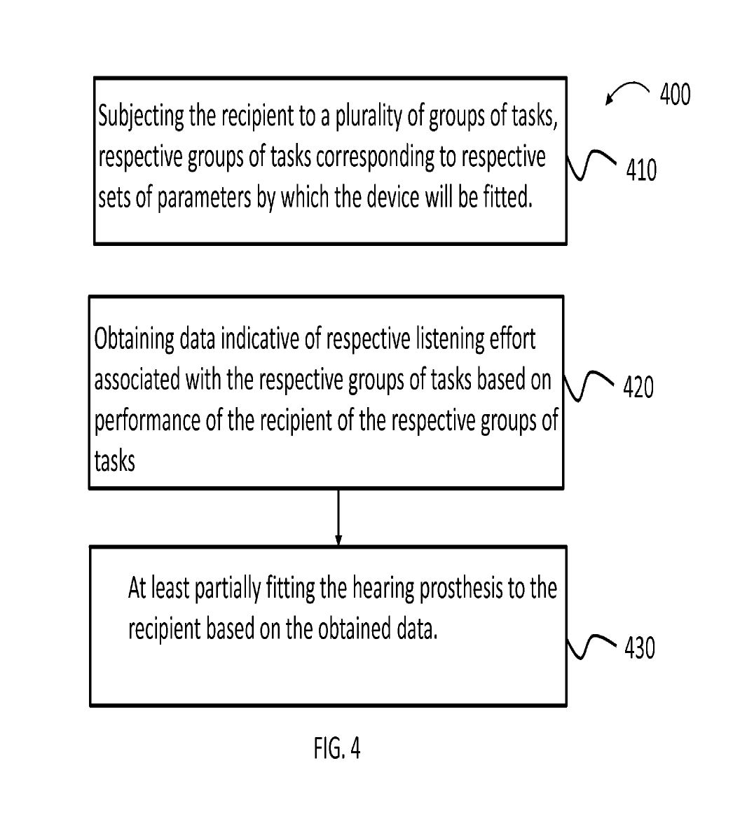

Thus, in view of the above, an exemplary embodiment includes method 400, which is represented by the flowchart of FIG. 4. Specifically, method 400 is a method of fitting a hearing prosthesis, such as a cochlear implant, to a recipient. Method 400 includes method action 410, which entails subjecting the recipient to a plurality of groups of tasks, respective groups of tasks corresponding to respective sets of parameters by which the device will be fitted. Additional details of method action 410 are detailed below. However, in an exemplary embodiment, respective groups of the groups of tasks entail method actions 210 and 220 detailed above, which are repeated for different respective sets of parameters. That said, in an alternative embodiment, method action 410 entails subjecting the recipient to respective groups of tasks where tasks of one group are drawn from a different cognitive domain than those of another group (e.g., the tasks of one group entail listening tasks and the task of another group entail visualization tasks, comprehension tasks and/or proprioceptive tasks, etc.).

Method 400 further includes method action 420, which entails obtaining data indicative of respective listening effort associated with the respective groups of tasks based on performance of the recipient of the respective groups of tasks. As with method action 410, additional details of method action 420 are presented below. However, it is briefly noted that in an exemplary embodiment, method action 420 entails obtaining the scores (and thus data) of the speech understanding tasks and the memory tasks of method actions 210 and 220, and evaluating those scores to determine a respective listening effort for a respective group of tasks of the groups of tasks. That said, in an alternative embodiment, method action 420 entails obtaining a ranking of listening effort that is based on the scores (and thus data indicative of respective listening effort).

Method 400 further includes method action 430, which entails at least partially fitting the hearing prosthesis to the recipient based on the data obtained in method action 420. By way of example only and not by way of limitation, an exemplary embodiment of method action 430 can entail identifying the group of tasks that corresponded to the highest level of ease of listening, identifying the corresponding set of parameters of the cochlear implant, and adjusting or otherwise setting the cochlear implant to evoke hearing percepts using those parameters (thus fitting the cochlear implant). Again, it is noted that in alternative embodiments, the set of parameters corresponding to the highest level of ease of listening may not be the set of parameters to which the cochlear implant is fitted. Other phenomenon may also be taken into account. Still, even if the set of parameters corresponding to the highest level of ease of listening is not selected, method action 430 can be executed as long as the ease of listening is taken into account.

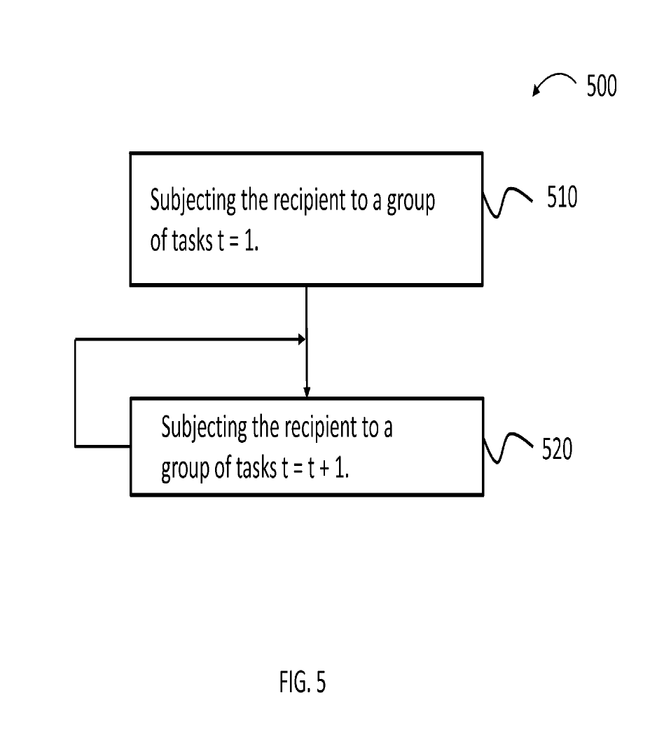

FIG. 5 depicts a flowchart for a method 500 which corresponds to method action 410 detailed above. Specifically, method 500 includes method action 510, which entails subjecting the recipient to a group of tasks t=1 (a first group of tasks). In an exemplary embodiment, the first group of tasks corresponds to executing method action 310 detailed above for set of parameters n=1. Method 500 further includes method action 520, which entails subjecting the recipient to a group of tasks t=t+1 (a second group of tasks). In an exemplary embodiment, the second group of tasks corresponds to executing method action 330 detailed above for set of parameters n=1+1. As can be seen from FIG. 5, method action 520 is repeated a number of times until the recipient is subjected to all of the groups of tasks. Accordingly, in an exemplary embodiment, groups of tasks to which the recipient is submitted respectively comprise (i) a listening task/speech perception task and (ii) a memory task based on information conveyed to the recipient during the listening task. In at least some embodiments, the groups of task will include a plurality of listening tasks and a plurality of memory tasks based on information conveyed to the recipient during the plurality of listening tasks. In this regard, method action 500 can be executed implementing the memory tasks and listening task detailed above and/or variations thereof. Accordingly, in embodiments where the groups of tasks entail memory tasks and listening tasks, the action of at least partially fitting the device to the recipient based on the data (method action 430) includes least partially fitting the device to the recipient based on data indicative of results of the listening tasks and the memory tasks of the respective groups.

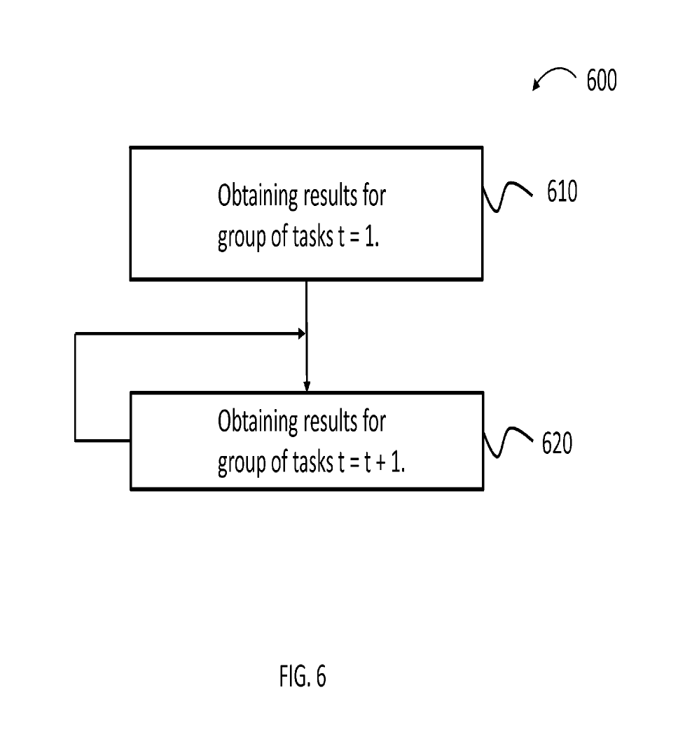

FIG. 6 depicts a flowchart for a method 600 which corresponds to method action 420 detailed above. Method 600 includes method 610, which entails obtaining results (which can be quantitative or otherwise) for the group of tasks t=1. In an exemplary embodiment, this corresponds to executing method action 320 detailed above for a set of parameters n=1. Method 600 further includes method action 620, which entails obtaining results (again, quantitative or otherwise) for the group of tasks t=t+1. In an exemplary embodiment, this corresponds to executing method action 340 detailed above for a set of parameters n=n+1. Thus, in an exemplary embodiment, this entails obtaining results of a listening task and obtaining results of a memory task. As can be seen from FIG. 6, method action 620 is repeated a number of times until respective quantitative results for the group of tasks are obtained (or at least desired quantitative results for a given group subtasks are obtained).

It is noted that method 400 can be implemented in a clinical setting with a clinician presenting material to the recipient (method action 410), the recipient giving feedback and the clinician scoring that feedback (method action 420). The clinician can print out a text copy of the sentences that he or she intends to present to the recipient, and can compare the feedback from the recipient to that text copy (both for the listening task and the memory task). By adequately noting the various text copies such that a correlation can be established between the text copies and a given set of parameters, the hearing prosthesis can be fitted to the recipient based on an evaluation of the notations of a given text copy (method action 430).

That said, alternate embodiments of method 400 can be executed utilizing more automated systems/devices. By way of example only and not by way of limitation, an exemplary embodiment could utilize a speaker system or the like that randomly produces speech sounds (sentences).

It is noted that in at least some embodiments, the dual task approach detailed above with respect to utilizing a listening task and a memory task can be relatively time-consuming, at least relative to some subjective tests. As noted above there are hundreds and/or even thousands of sets of parameter settings for at least some hearing prostheses, such as cochlear implants. By way of example only and not by way of limitation, utilizing the above-noted dual task approach for measuring the speech processing and listening effort scores for all possible cochlear implant programs or parameter combinations could take many hours, days or even longer. Further, in at least some embodiments, the tasks presented in the dual task approach might be relatively fatiguing to the recipient. Thus, an exemplary embodiment can include utilizing the teachings detailed herein and/or variations thereof in combination with other types of methods to streamline the fitting process. By way of example only and not by way of limitation, subjective processes can be utilized in combination with the objective processes detailed herein to streamline the fitting process. Specifically, subjective processes can be utilized to reduce the sets of conditions to a number that is utilitarian for the objective processes detailed herein (e.g., the dual task approach) to be utilized. That is, subjective processes can be used to “vet” the tens, hundreds, or even thousands of parameter sets to a “manageable” or otherwise more utilitarian number, upon which the objective tasks detailed herein will be based.

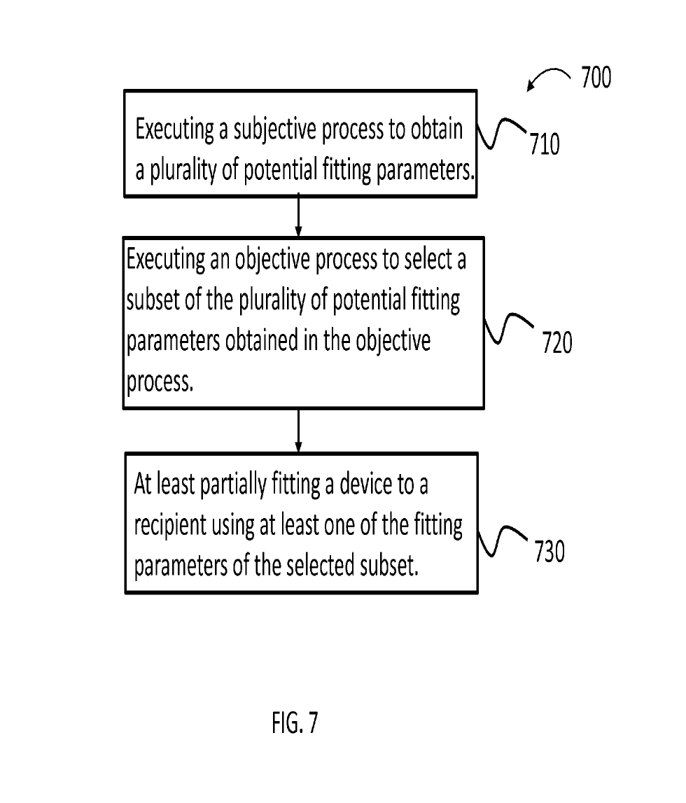

Along these lines, referring now to FIG. 7, there is a method 700 that utilizes both subjective processes and objective processes to fit the hearing prosthesis. More specifically method 700 includes method action 710, which entails executing a subjective process to obtain a plurality of potential fitting parameters/a plurality of sets of fitting parameters. In an exemplary embodiment, the fitting parameters/sets of fitting parameters are fitting parameters of a cochlear implant or other type of hearing prostheses. By “potential,” it is meant that a subsection of the various potential fitting parameters/sets of parameters (from amongst the tens, hundreds and/or thousands of such) are identified, 2, 3, 4, 5, 6, 7, 8, 9, 10 or more various potential fitting parameters all of which are candidates to be applied in the ultimate fitting action, one of which will be ultimately applied in the ultimate fitting action.

In an exemplary embodiment, method action 710 can be executed by identifying two different parameters, the combinations of which can form a matrix. In an exemplary embodiment, an audiologist or the like presents short processed audio examples using each condition from the matrix to the recipient, and allowing the recipient to judge whether that particular condition is worthy enough to continue on to further assessment (e.g., thorough an objective process, as will be detailed below). In alternative embodiment, such as with respect to application of a cochlear implants fitting which could require the assessment of combinations constituting hundreds or even thousands of processor settings, by way of example only and not by way of limitation, a genetic algorithm process can be used, such as algorithms detailed in the teachings of U.S. patent application publication number 2010/0152813 to Dr. Sean Lineaweaver, filed on Sep. 10, 2009.

More specifically, in at least some embodiments, the methods detailed herein are executed utilizing a medical device, such as the cochlear implant detailed above, where there can be hundreds or thousands of possible parameter map sets (e.g., more than 100, more than 500, more than 1,000, more than 1,500, more than 2,000). In some embodiments, the device has any value or range of values of sets of parameters between 10 and 3,000 or more in 1 increment (e.g., more than 123, 502-1007, more than 2222, etc.). It can be, in at least some embodiments, impractical for a recipient to experience all of the alternatives utilizing the dual task approach detailed herein. It is also difficult, but not impossible, to identify a set of parameters by prescription based on a limited set of measurements as is, for example, the case in fitting eyeglasses. Because parameters of cochlear implant systems often interact non-linearly and non-monotonically, it is also not possible to sequentially “optimize” parameters one at a time, adjusting each in succession to its optimal value. Accordingly, an exemplary embodiment entails executing method action 710 by utilizing one or more or all of the teachings of the just-noted patent application publication to reduce (e.g., rapidly reduce) hundreds and/or thousands of processor programs/sets of parameters into a group of two, three, four, five, six, seven, eight, nine, ten, eleven and/or twelve or more that are deemed utilitarian as a result of the process.

Accordingly, an exemplary embodiment of method action 710 entails obtaining the potential fitting parameter sets from a group comprising at least 30, 40, 50, 60, 70, 80, 90, 100, 120, 140, 160, 180, 200, 225, 250, 275, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100 or more sets of parameters, where the parameters correspond to respective processor programs with which the device (e.g., cochlear implant) can be programmed, and thus configured.

Subsequent to executing method action 710, method 720 is executed, which entails executing an objective process to select a subset of the plurality of potential fitting sets of parameters obtained in the subjective process (method action 710). By way of example only and not by way of limitation, method action 720 can be accomplished by executing method actions 310, 320, 330 and 340 detailed above. By way of example, method action 720 can be executed using any of the dual task approaches as detailed herein and/or variations thereof.

Still with reference to FIG. 7, method 700 further includes method action 730, which entails at least partially fitting a device (e.g., a cochlear implant or other type of hearing prosthesis) to the recipient using the selected subset selected in method action 720.

As noted above, some and/or all of the teachings detailed herein and variations thereof can be implemented utilizing automation. In an exemplary embodiment, any or all of the methods detailed herein and/or variations thereof can be practiced in an automated fashion. Indeed, as noted above, at least some of the method actions detailed herein can be practiced utilizing an interactive automated process or the like.

In view of the above, it can be seen that in an exemplary embodiment entails a method of fitting a hearing prosthesis or other type of medical device where the assessments of various sets of parameters with which the device will be fitted is not done on a continual basis. In this regard, one or more or all of the above methods are executed during a fitting session or during a plurality of fitting sessions, and subsequently, the recipient goes off and utilizes the medical device (e.g. utilizes the hearing prosthesis to evoke hearing percepts). It is also noted that in at least some embodiments, one or more or all of the method actions detailed herein are executed without regard to sound quality or the like. Additionally, at least some embodiments are implemented where one or more or all of the method actions detailed herein are executed utilizing comparisons between more than two candidate sets, at least at one instance. For example, the genetic algorithm detailed above results in comparisons being made between more than two candidate sets of parameters.

An exemplary system and an exemplary device/devices that can enable the teachings detailed herein, which in at least some embodiments can utilize automation, will now be described in the context of a recipient operated fitting system. That is, an exemplary embodiment includes executing one or more or all of the methods detailed herein and variations thereof, at least in part, by a recipient.

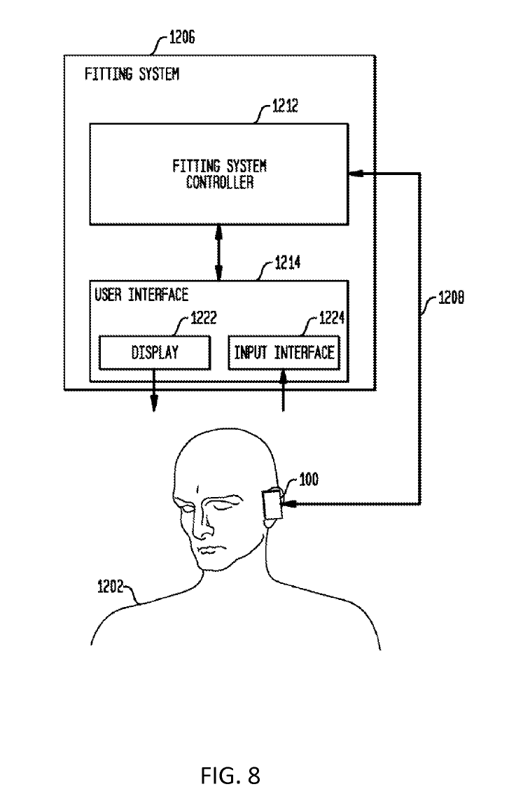

FIG. 8 is a schematic diagram illustrating one exemplary arrangement in which a recipient 1202 operated fitting system 1206 can be used in fitting a medical device, such as cochlear implant system 100. In the embodiment illustrated in FIG. 8, the cochlear implant system can be directly connected to fitting system 1206 to establish a data communication link 1208 between the speech processor 116 and fitting system 1206. Fitting system 1206 is thereafter bi-directionally coupled by a data communication link 1208 with speech processor 116. While the embodiment depicted in FIG. 8 depicts a fitting system 1206 and a hearing prosthesis connected via a cable, any communications link that will enable the teachings detailed herein that will communicably couple the implant and fitting system can be utilized in at least some embodiments.

Fitting system 1206 can comprise a fitting system controller 1212 as well as a user interface 1214. Controller 1212 can be any type of device capable of executing instructions such as, for example, a general or special purpose computer, a handheld computer (e.g., personal digital assistant (PDA)), digital electronic circuitry, integrated circuitry, specially designed ASICs (application specific integrated circuits), firmware, software, and/or combinations thereof. As will be detailed below, in an exemplary embodiment, controller 1212 is a processor. Controller 1212 can further comprise an interface for establishing the data communications link 1208 with the device 100 (e.g., cochlear implant 100). In embodiments in which controller 1212 comprises a computer, this interface may be for example, internal or external to the computer. For example, in an embodiment, controller 1206 and cochlear implant may each comprise a USB, Firewire, Bluetooth, WiFi, or other communications interface through which data communications link 1208 may be established. Controller 1212 can further comprise a storage for use in storing information. This storage can be for example, volatile or non-volatile storage, such as, for example, random access memory, solid state storage, magnetic storage, holographic storage, etc.

User interface 1214 can comprise a display 1222 and an input interface 1224. Display 1222 can be, for example, any type of display device, such as, for example, those commonly used with computer systems. In an exemplary embodiment, element 1222 corresponds to a device configured to visually display a plurality of words to the recipient (which includes sentences), as detailed above.

Input interface 1224 can be any type of interface capable of receiving information from a patient, such as, for example, a computer keyboard, mouse, voice-responsive software, touch-screen (e.g., integrated with display 1222), microphone (e.g. optionally coupled with voice recognition software or the like) retinal control, joystick, and any other data entry or data presentation formats now or later developed. It is noted that in an exemplary embodiment, display 1222 and input interface 1224 can be the same component, e.g., in the case of a touch screen). In an exemplary embodiment, input interface 1224 is a device configured to receive input from the recipient indicative of a choice of one or more of the plurality of words presented by display 1222.

In an exemplary embodiment, user interface 1214 is configured to present to the recipient at least one of a visual, a language or a proprioceptive stimulation. In an exemplary embodiment, the visual task can be a reaction task (e.g., the system can direct a laser pointer at an object, and the recipient identifies the occurrence of such, etc.) In an exemplary embodiment, the language task is comprehension of the correctness of a sentence (e.g., a sentence such as the dog had a loud bark vs. the dog had a loud meow, etc.). In an exemplary embodiment, the proprioceptive task is the identification of a body portion to which stimulation is applied (or simply that the body has been stimulated). Other non-listening tasks can include tasks that distract the recipient from listening (e.g., presentation of visually appealing or unappealing picture or video, sounds of fingernails on a chalkboard or sound of a favorite actor or actress of the recipient, etc.). It is noted that some embodiments can be implemented utilizing a dual task approach where the tasks are drawn from different cognitive domains irrespective of whether the systems detailed herein and/or variations thereof are utilized. Indeed, any task that can influence the ability of ease of listening can be utilized in at least some embodiments. In some embodiments, this can be the case when the tasks are presented in close temporal proximity to one another (e.g., simultaneously, within a half second of one another, within a second of one another, within about 2, 3, 4, 5 seconds of one another, etc.).

It is noted that in at least some embodiments of the teachings detailed herein, the actions of subjecting the recipient to different tasks of a different type have at least parallels to situations in which the recipient will be exposed to during normal use of the hearing prosthesis. By way of example only and not by way of limitation, the user will often be in a situation where he or she is trying to listen but is also distracted, such as by a visual image. Still further by way of example only and not by way of limitation, the user will often experience tactile stimulation while listening with the hearing prosthesis. Accordingly, in an exemplary embodiment, the teachings detailed herein can be used to help acclimate the recipient to a normal listening environment (as opposed to be controlled environment of a traditional fitting session). Corollary to this is that in an exemplary embodiment, the teachings detailed herein can be used to provide an environment in which the hearing prosthesis is fitted to the recipient that more closely corresponds to an environment in which the recipient will find himself or herself. That is, the hearing prosthesis will be fitted to the recipient based on results that more closely correspond to actual listening experiences of the recipient, or at least more difficult listening experiences. Indeed, exemplary embodiments can be used to train the recipient to hear better during difficult listening environments (e.g., those where there are distractions), and can be used to fit the hearing prosthesis for use in more difficult listening environments (the idea being that even if the fitting is not optimized for the average listening environment, the listening experience will still be better because the difficult listening experiences will not be as difficult, even though perhaps the average listening experiences may be more difficult, all relative to that which would be the case in the absence of the teachings detailed herein and are variations thereof).

In view of the above, it is noted that in at least some embodiments, some of the tasks entail tasks that the recipient will experience during normal listening scenarios. Such task can be routine tasks.

Accordingly, in an exemplary embodiment, the system is configured to present to the recipient an audible sentence including a word included in the plurality of words in synchronization with the presentation to the recipient of the at least one of a visual, a language or a proprioceptive stimulation. The information pertaining to word perception is based on the presented audible sentence.

Processor 1212 is configured to receive information indicative of the input from the recipient and determine whether the choice corresponds to one or more words in a sentence previously presented to the recipient. In an exemplary embodiment, the one or more words corresponds to fewer words than that in the sentence previously presented to the recipient. In an exemplary embodiment, the received information indicative of the input from the recipient is information pertaining to the memory task detailed herein. Processor 1212 is further configured to select a fitting parameter based on the determination and based on information pertaining to word perception of the sentence previously presented to the recipient. In an exemplary embodiment, processor 1212 is configured to control the system of FIG. 8 to execute one or more or all of the method actions detailed herein and are variations thereof.

In an exemplary embodiment, system 1206 is further configured to present to the device 100 (e.g., cochlear implant 100) and audible sentence including a word included in the plurality of words. An exemplary embodiment, the audible sentence corresponds to the sentence previously presented to the recipient. By “audible sentence,” it is meant a sentence that evokes a hearing percept by the hearing prosthesis 100. An exemplary embodiment, system 1206 includes a speaker or the like which generates an acoustic signal corresponding to the audible sentence that is picked up by a microphone of the hearing prosthesis 100. In an alternate embodiment, system 1206 is configured to provide a non-acoustic signal (e.g., an electrical signal) to the hearing prosthesis processor by bypassing the microphone thereof, thereby presenting an audible sentence to the hearing prosthesis. It is noted that in an exemplary embodiment, the information pertaining to word perception is based on the presented audible sentence. Along these lines, in an exemplary embodiment, the system 1206 is configured to receive input from the recipient indicative of a perceived sentence in response to presentation of the audible sentence, thus enabling the teachings detailed above with respect to providing a recipient the ability to select from a plurality of sentences presented on a video screen of the like. In an exemplary embodiment, this can be achieved via the input interface 1224. More specifically, as detailed above, a touchscreen or the like can be utilized as input interface 1224. Accordingly, in an exemplary embodiment, the system 1206 is configured to visually display a plurality of sentences to the recipient, where at least one of the plurality of sentences displayed to the recipient corresponds to the audible sentence. In this exemplary embodiment, the system 1206 is configured to receive input from the recipient indicative of a choice of one of the plurality of sentences. That said, in an alternate embodiment, a microphone of the like can be utilized to receive vocalized input from the recipient. When coupled with speech recognition software or otherwise automated speech recognition algorithm or the like, the recipient's audible responses can be utilized as input from the recipient indicative of a perceived sentence. Any device, system and/or method that is configured to receive input from the recipient can be utilized in at least some embodiments.

It is further noted that in at least some embodiments, the speech recognition algorithm can be coupled with a feedback system that presents information to the recipient indicative of what the speech recognition algorithm perceived as being spoken by the recipient. In this manner, the recipient can be provided with an indication of what the system perceived as being spoken, and can correct the system with respect to what the recipient actually said if there is a misperception (e.g., by the recipient repeating the words, the recipient typing in the actual words, etc.).

An exemplary embodiment, processor 1212 is configured to evaluate the received input for congruence between the perceived sentence and the audible sentence. In an exemplary embodiment, this entails comparing the sentence that the recipient touched on the touchscreen to the sentence forming the basis of the audible sentence. In an alternate exemplary embodiment this entails comparing data from speech recognition software based on the recipient's response captured by microphone with the sentence forming the basis of the audible sentence.

As noted above, some embodiments detailed herein decouple accuracy in the listening tasks from the memory tasks. Accordingly, in an exemplary embodiment, the system 1206 is configured to make a determination whether the choice corresponds to one or more words in a sentence previously presented to the recipient based on a result of the evaluation of the received input indicative of the perceived sentence. Thus, in an exemplary embodiment, the received input from the recipient indicative of the choice of one of the plurality of sentences corresponds to the input from the recipient indicative of the perceived sentence. That is, in an exemplary embodiment, system 1212 is configured to take into account the fact that the recipient may have incorrectly perceived one or more words in a sentence presented to him or her during the listening test, and based the memory test on what the recipient perceived as opposed to the actual word presented to the recipient.

In view of the above, an exemplary embodiment includes the system 1206 configured with a processor 1212 that is configured to at least partially fit the device 100 based on data based on listening effort, wherein the data based on listening effort is based on the determination of whether the choice corresponds to one or more words in a sentence previously presented to the recipient.

Still further, as noted above, at least some embodiments include utilization of both subjective and object of tasks to develop a data set to fit the medical device. Accordingly, in an exemplary embodiment, the system 1206 is configured to execute a genetic algorithm to select a determined value set comprising values for a plurality of fitting parameters. The genetic algorithm can be in accordance with that detailed above and/or variations thereof. The system is further configured to utilize the genetic algorithm in combination with the determination of whether the choice corresponds to one or more words in a sentence previously presented to the recipient and the information pertaining to word perception to identify a set of parameters and fit the device using that identified set of parameters.

It is noted that the system 1206 detailed above can execute one or more or all of the actions detailed herein and are variations thereof automatically, at least those that do not require the inputs of the recipient. It is noted that the schematic of FIG. 8 is functional. In some embodiments, a fitting system 1206 is a self-contained device (e.g. a laptop computer) that is configured to execute one or more or all of the method actions detailed herein and are variations thereof, aside from those that utilize the recipient and/or the audiologist without receiving input from an outside source. In an alternative embodiment, fitting system 1206 is a system having components located at various geographical locations. By way of example only and not by way of limitation, user interface 1214 can be located with the recipient, and the fitting system controller (e.g., processor) 1212 can be located remote from the recipient. By way of example only and not by way limitation, the system controller 1212 can communicate with the user interface 1214 via the Internet and/or via cellular communication technology or the like. Indeed, in at least some embodiments, the system controller 1212 can also communicate with the device 100 via the Internet and/or via cellular communication or the like. In an exemplary embodiment, the user interface 1214 can be a portable communications device, such as by way of example only and not by way of limitation, a cell phone and/or a so-called smart phone. Indeed, user interface 1214 can be utilized as part of a laptop computer or the like. Any arrangement that can enable system 1206 to be practiced and/or that can enable a system that can enable the teachings detailed herein and/or variations thereof to be practiced can be utilized in at least some embodiments.

It is further noted that in at least some embodiments, the system 1206 can enable the teachings detailed herein and are variations thereof to be practiced at least without the direct participation of a clinician (e.g. an audiologist). Indeed, in at least some embodiments, the teachings detailed herein and/or variations thereof, at least some of them, can be practiced without the participation of a clinician entirely. In an alternate embodiment, the teachings detailed herein and/or variations thereof, at least some of them, can be practiced in such a manner that the clinician only interacts otherwise involves himself or herself in the process to verify that the results are acceptable or otherwise that desired actions were taken. In the above, it is noted that in at least some embodiments, a computerized automated application can be implemented to score or otherwise determine the results of the tasks detailed herein (e.g. listening task and or memory task).

It is noted that any method detailed herein also corresponds to a disclosure of a device and/or system configured to execute one or more or all of the method actions associated there with detailed herein. In an exemplary embodiment, this device and/or system is configured to execute one or more or all of the method actions in an automated fashion. That said, in an alternate embodiment, the device and/or system is configured to execute one or more or all of the method actions after being prompted by the recipient and/or by the clinician.

It is noted that embodiments include non-transitory computer-readable media having recorded thereon, a computer program for executing one or more or any of the method actions detailed herein. Indeed, in an exemplary embodiment, there is a non-transitory computer-readable media having recorded thereon, a computer program for executing at least a portion of a method of fitting a hearing prosthesis to a recipient, the computer program including code for obtaining data indicative of respective listening effort by the recipient associated with a respective groups of tasks subjected to the recipient based on performance of the recipient of the respective groups of tasks, the respective groups of tasks corresponding to respective sets of parameters by which the device will be fitted, and code for at least partially fitting the hearing prosthesis to the recipient based on the obtained data.

In an exemplary embodiment, the aforementioned non-transitory computer readable medium is such that the groups of tasks to which the recipient is submitted respectively comprise a first group of tasks, and a second group of tasks of a different type than the tasks of the first group, wherein the tasks of the first group are drawn from, in some embodiments, a different cognitive domain of the recipient than those of the second group, and in some embodiments, the same cognitive domain of the recipient as those of the second group. Thus, in an exemplary embodiment, the groups of tasks to which the recipient is submitted respectively comprise listening task(s), and task(s) drawn from different cognitive domain(s) than that of the listening task(s), while in other embodiments, the groups of tasks to which the recipient is submitted respectively comprise listening task(s), and tasks drawn from the same cognitive domain as that of the listening task(s). More specifically, in an exemplary embodiment, the groups of tasks to which the recipient is submitted respectively comprise listening task(s) and at least one of visual task(s), comprehension task(s) or proprioceptive task(s), while in other embodiments, the groups of tasks to which the recipient is submitted respectively comprise listening task(s) and memory tasks(s).

It is further noted that any device and/or system detailed herein also corresponds to a disclosure of a method of operating that device and/or using that device. Furthermore, any device and/or system detailed herein also corresponds to a disclosure of manufacturing or otherwise providing that device and/or system.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A method of at least partially fitting a hearing prosthesis device to a recipient, comprising:
subjecting the recipient to a first task;
subjecting the recipient to a second task of a different type than the first task, wherein the first task and the second task draw from the same cognitive domain of the recipient; and
at least partially fitting the device to the recipient based on results of the first and second task.

2. The method of claim 1, further comprising:
obtaining the results by assessing results of the first task and the second task based on quantitative scoring.

3. The method of claim 1, wherein:
the first task is a speech understanding task.

4. The method of claim 3, wherein:
the first task is a sentence recognition test.

5. The method of claim 4, wherein:
a plurality of sentences are presented to the recipient; and
subsequent to presentation of respective sentences, the recipient identifies what the recipient perceived as the respective sentence.

6. The method of claim 1, wherein:
the second task is a memory task.

7. The method of claim 6, wherein:
a plurality of sentences are presented to the recipient; and
subsequent to presentation of the plurality of sentences, the recipient identifies what the recipient perceived as the respective last word of the respective sentences of the plurality of sentences.

8. The method of claim 2, further comprising:
determining a listening effort based on the obtained results; and
at least partially fitting the device to the recipient based on ease of listening.

9. The method of claim 1, wherein:
the device includes a cochlear implant, wherein the action of fitting the device to the recipient includes fitting the cochlear implant to the recipient.

10-23. (canceled)

24. A system for at least partially fitting a device to a recipient, comprising:
a processor; and
a device configured to visually display a plurality of words to the recipient, wherein
the system is configured to receive input from the recipient indicative of a choice of one or more of the plurality of words,
the processor is configured to receive information indicative of the input from the recipient and determine whether the choice corresponds to one or more words in a sentence previously presented to the recipient, and
the processor is configured to select a fitting parameter based on the determination and based on information pertaining to word perception of the sentence previously presented to the recipient.

25. The system of claim 24, wherein
the one or more words corresponds to fewer words than that in the sentence previously presented to the recipient.

26. The system of claim 24, wherein:
the system is configured to present to the recipient an audible sentence including a word included in the plurality of words; and
the information pertaining to word perception is based on the presented audible sentence.

27. The system of claim 25, wherein:
the system is configured to receive input from the recipient indicative of a perceived sentence in response to presentation of the audible sentence;
the system is configured to evaluate the received input for congruence between the perceived sentence and the audible sentence;
the system is configured to make the determination whether the choice corresponds to one or more words in a sentence previously presented to the recipient based on a result of the evaluation of the received input indicative of the perceived sentence,
the system is configured to visually display a plurality of sentences to the recipient;
at least one of the plurality of sentences corresponds to the audible sentence;
the system is configured to receive input from the recipient indicative of a choice of one of the plurality of sentences;
the received input from the recipient indicative of the choice of one of the plurality of sentences corresponds to the input from the recipient indicative of the perceived sentence; and
the device configured to visually display a plurality of words to the recipient is a video display screen configured to receive touch input corresponding to the input from the recipient.

28-30. (canceled)

31. The system of claim 24, wherein:
the system is configured to execute a genetic algorithm to select a determined value set comprising values for a plurality of fitting parameters; and
the system is configured to utilize the genetic algorithm in combination with the determination of whether the choice corresponds to one or more words in a sentence previously presented to the recipient and the information pertaining to word perception to identify a set of parameters and fit the device using that identified set of parameters.

32-33. (canceled)

34. A method, comprising:
executing a subjective process to obtain a plurality of potential fitting parameters; and
after executing the subjective process, executing an objective process to select a subset of the plurality of potential fitting parameters obtained in the subjective process;
at least partially fitting a device to a recipient using at least one of the fitting parameters of the selected subset.

35. The method of claim 34, wherein the objective process includes:
identifying first fitting parameters that are, for the recipient, indicative of least effortful listing relative to other fitting parameters;
identifying second fitting parameters that are, for the recipient, indicative of most perceivable speech relative to other fitting parameters; and
selecting the subset based on the identification of the first fitting parameters and the second fitting parameters.

36. The method of claim 35, wherein the action of selecting the subset based on the identification includes:
selecting one or more of the first fitting parameters and selecting one or more of the second fitting parameters to establish the selected subset.

37. The method of claim 34, wherein the objective process includes:
identifying fitting parameters that are, for the recipient, indicative of least effortful listing relative to other fitting parameters and indicative of most perceivable speech relative to other fitting parameters; and
selecting the subset based on the identified fitting parameters.

38. (canceled)

39. The method of claim 34, wherein:
the subjective process includes utilizing a genetic algorithm to obtain the plurality of potential fitting parameters.

40. The method of claim 34, wherein:
the parameters correspond to respective processor programs with which the device can be programmed; and
the obtained plurality of potential fitting parameters are obtained from a group comprising at least one hundred parameters.

41. (canceled)
